(19) United States
(12) Patent Application Publication
(10) Pub. No.: US 2002/0123702 A1
(43) Pub. Date: Sep. 5, 2002

(54) Method & apparatus for mitigating renal failure using mechanical vibration including ultrasound and heat

(76) Inventor: Young Cho, Cherry Hill, NJ (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/082,660

(22) Filed: Feb. 25, 2002

Related U.S. Application Data
(60) Provisional application No. 60/271,558, filed on Feb. 26, 2001.

(51) Int. Cl. A61N 7/00

(52) U.S. Cl. 601/2

(57) ABSTRACT

A method and apparatus for preventing or at least mitigating renal failure due to the presence of contrast media agents administered to patients. The method and apparatus applies either mechanical vibration, including ultrasonic energy, or heat to the vicinity of the kidneys at the patient's back to reduce the blood viscosity of the blood in the microvessels of the kidney.

### RELATED APPLICATIONS

This application is an Application based on Provisional Application Serial No. 60/271,558 filed Feb. 26, 2001 entitled METHOD & APPARATUS FOR MITIGATING RENAL FAILURE USING MECHANICAL VIBRATION INCLUDING ULTRASOUND, and whose entire disclosure is incorporated by reference herein.

### FIELD OF THE INVENTION

The invention pertains to methods and apparatus for reducing blood viscosity of living beings, and more particularly, to methods and apparatus for preventing renal failure due to the administration of contrast media agents to a living being's blood.

### BACKGROUND OF INVENTION

With the use of such devices as magnetic resonance imaging (MRI) apparatus, there is a need to administer contrast media (CM) agents (for example, Acetrizoate, Iothalamate, Metrizamide, Iopamidol, Iodixanol, oral cholecystographic agents such as Iopanoic acid, Ipodate sodium, GI contrast agents such as Diatrizoate sodium, parenteral agents such as Diatrizoate Meglumine and any salts or combinations thereof) to the patient undergoing the MRI. For example, if a brain scan is being conducted using the MRI apparatus, the presence of the CM agent in the patient's bloodstream permits good tracking results. However, it has been well-documented that in certain numbers of patients, the administering of the CM agent to the patient causes renal failure that could ultimately result in the loss of the kidney.

Such renal failure is believed to be caused by an increased blood viscosity. Increased blood viscosity reduces blood perfusion in the kidneys, thus starting blood clotting and eventual acute renal failure. FIG. 1 is a diagram of a kidney of an adult human being; the physical dimensions of the kidney are approximately: 10-12 cm (4-5 in) in length, 5-7.5 cm (2-3 in) in width and 2.5 cm (1 in) in thickness; FIG. 2 depicts the relative positions of the kidneys in a human being. In particular, the kidney comprises microvessels. It is believed that the CM agent alters the blood viscosity by increasing low shear viscosity through these microvessels. As blood flow decreases, the apparent blood viscosity increases due to non-Newtonian characteristics of whole blood; see FIG. 3. As the blood is thickened by the contrast media agents, blood flow in the nephron (a functional unit of the kidney) is reduced. This causes poor blood perfusion that can lead to thrombosis and eventual blood clotting. This can result in acute renal failure, and total kidney loss.

Therefore, there remains a need to prevent or at least mitigate the reduction of such blood perfusion when a CM agent has been administered to patient, and thereby prevent acute renal failure and the loss of a kidney.

### SUMMARY OF THE INVENTION

A method for preventing, or reducing the likelihood of, renal failure in a living being due to the presence of a contrast media agent in the bloodstream of the being. The method comprises the step of reducing the viscosity of the blood in the kidney.

An apparatus for preventing, or reducing the likelihood of, renal failure in a living being due to the presence of a contrast media agent in the bloodstream of the being. The apparatus comprises a vibrator for producing vibratory energy when energized and wherein the apparatus is locatable with respect to the body of the being to provide the vibratory energy to the being to reduce the viscosity of the blood in the being's kidney.

An apparatus for preventing, or reducing the likelihood of, renal failure in a living being due to the presence of a contrast media agent in the bloodstream of the being. The apparatus comprises a heater for producing heat energy when energized and wherein the apparatus is locatable with respect to the body of the being to provide the heat energy to the being to reduce the viscosity of the blood in the being's kidney.

### DESCRIPTION OF THE DRAWINGS

FIG. 1 is a diagrammatic view of a kidney of an adult human being;

FIG. 2 is a cross-sectional view of a human being through the torso, at T12 of the vertebral body, showing the relative positions of the kidneys;

FIG. 3 is a graphical depiction of the relationship between blood viscosity and shear rate;

FIG. 4 is a block diagram of a first embodiment of the present invention;

FIG. 5 is a graphical depiction of a frequency range that was used in laboratory testing using the present invention for reducing the blood viscosity of a human being;

FIG. 6 is a log-log graphical depiction of the relationship between blood viscosity and temperature;

FIG. 7 is a regular graphical depiction of the relationship between blood viscosity and temperature; and

FIG. 8 is a block diagram of a second embodiment of the present invention.

### DETAILED DESCRIPTION OF THE PREFERRED EMBODIMENTS

The present invention uses mechanical vibration located at the back of a human being to reduce whole blood viscosity such that the blood perfusions in afferent arteriole and efferent arteriole increase, thus preventing the formation of thrombosis. In particular, blood flow at the glomerulus is very slow and a small increase in blood viscosity due to the presence of the CM agent may adversely affect blood perfusion. Mechanical vibration helps the blood to keep flowing by reducing its viscosity.

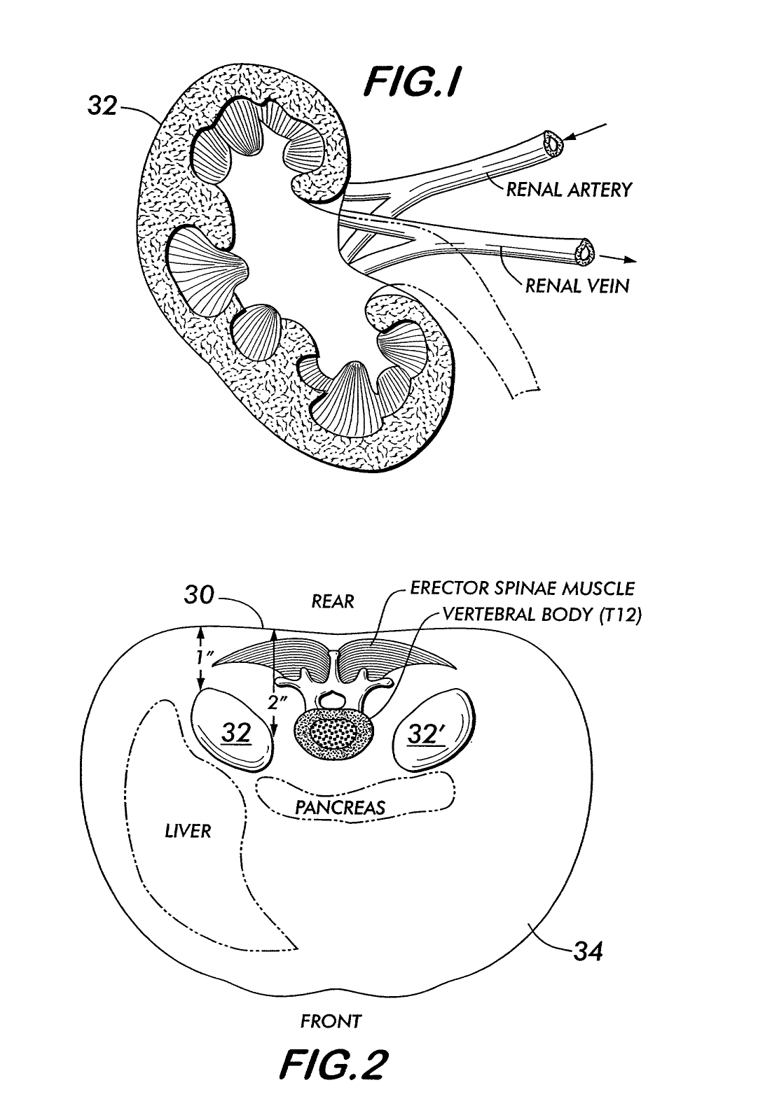

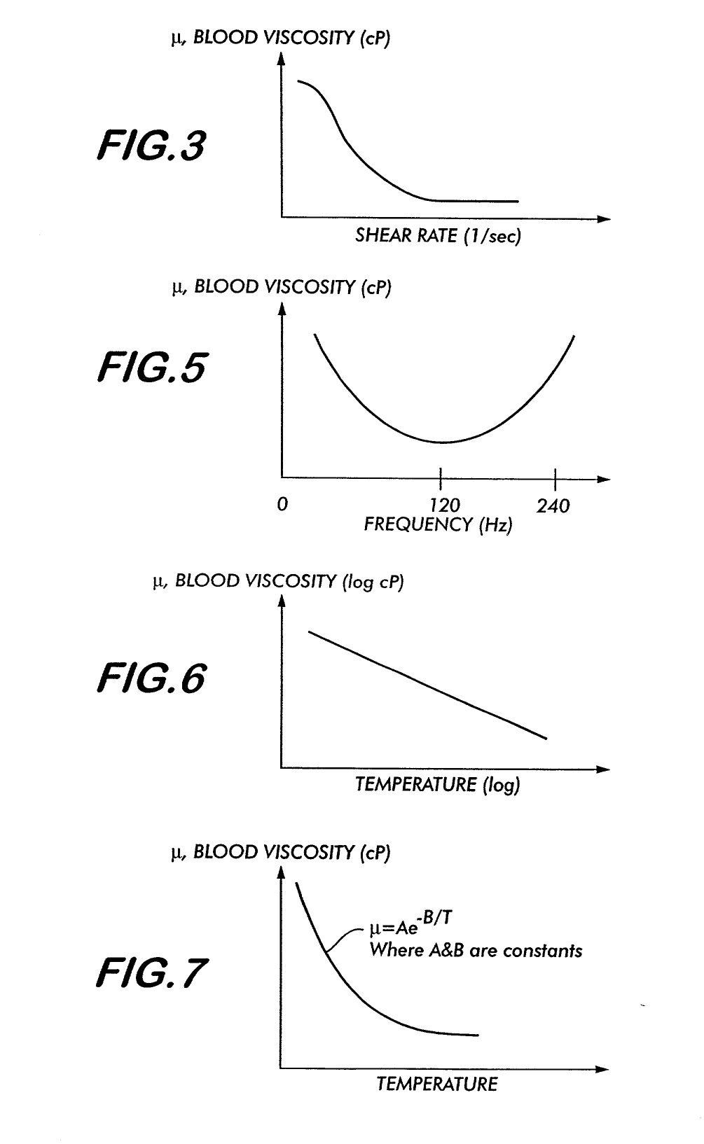

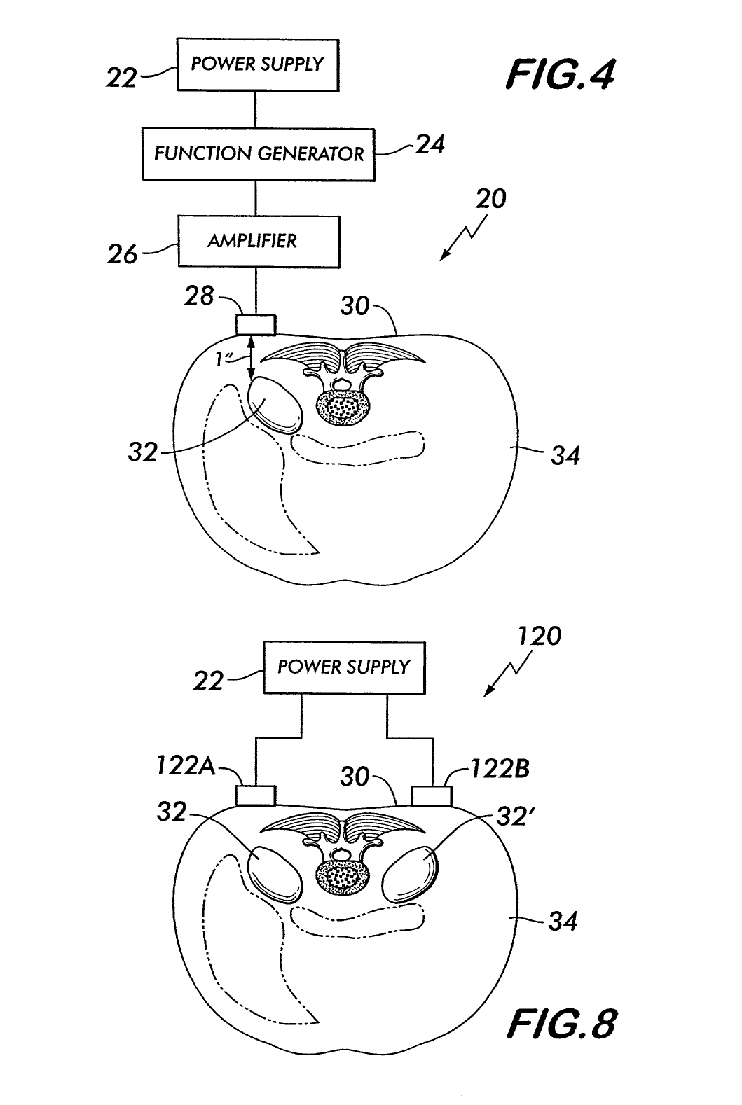

Referring now in detail to the various figures of the drawing wherein like reference characters refer to like parts, there is shown at 20 in FIG. 4 an apparatus for preventing renal failure. In particular, the apparatus 20 comprises a power supply 22, a function generator 24, an amplifier 26 and a mechanical vibrator 28. The vibrator 28 is applied to the back side of a patient in the vicinity of the kidney; e.g., as shown in FIG. 4, the vibrator 28 (e.g., mechanical vibrator from Bruel & Kjaer; model 8202) has been applied to the back side 30 of a patient 34 in the vicinity of the right kidney 32. The mechanical vibrator 28 is large enough (e.g., 4-5 inches in diameter, and comprising a flat disk shape) to cover the whole kidney. As also shown in FIG. 2, there is approximately 1 inch separation between the back of the patient and the right kidney. It should be understood that although only one mechanical vibrator 28 is shown for application to one kidney, a pair of mechanical vibrators could be used for application to a respective kidney.

It should be understood that the term “mechanical vibrator” 28 encompasses any device that can emit mechanical vibration and includes ultrasound devices that operate at frequencies above 20 kHz. The mechanical vibrator 28, by emitting such vibratory energy, shakes any thrombosis and breaks any blood clots that have formed due to the presence of the CM agent. Thus, the mechanical vibrator 28 acts to alter the blood rheology and thereby prevent thrombosis and clotting.

For example, using the apparatus 20 in laboratory testing has determined that whole blood viscosity can be reduced substantially at a particular frequency of 110-120 Hz. In particular, during the laboratory testing, the apparatus 20 used a frequency of 120 Hz; see FIG. 5 . However, as stated previously, it is within the broadest scope to include a wide range of vibration frequencies, including the ultrasonic range.

Furthermore, since blood viscosity decreases with increasing temperature (see FIGS. 6-7 which depict this relationship in a log-log graph and a regular graph, respectively), another embodiment of the present invention, namely apparatus 120 as shown in FIG. 8, utilizes localized heating (not very intense heat) to just reduce the blood viscosity near and around the kidney. This improves blood flow in the kidney, thereby preventing or at least mitigating blood clotting and renal failure. In particular, the apparatus 120 comprises the power supply 22 (e.g., power supply from B&K Precision; model 1689) and a pair of heating elements 128A and 128B (e.g., heating components from Watlow; model VF504A18S) that, like the mechanical vibrator 28, covers a respective kidney 32 and 32′. These heating elements 128A/128B increase the temperature of the blood entering the respective kidney.

Thus, in view of the above discussion, it should be understood that the present invention comprises the application of mechanical vibration, including ultrasound, or heat to the kidneys to a patient that may be susceptible to renal failure due to the administration of a CM agent. The vibratory energy shakes any thrombosis or breaks up any blot clots that may have formed in the kidneys due to the presence of the CM agent in the bloodstream. Similarly, because the presence of the CM agent in the bloodstream tends to increase the low shear viscosity in the microvessels of the kidneys which causes thrombosis and blood clotting, and because blood viscosity is inversely related to temperature, the application of heat to the kidneys also acts to reduce the viscosity of the blood and thereby prevent thrombosis or blood clotting.

It should be understood that the apparatus 20 and 120 can be portable such that they can be used in a catheter laboratory or at any other location. For example, the mechanical vibrator 28, or the heating elements 128A/128B, can be secured to a table in the catheter laboratory and upon which the patient can lie. Alternatively, the mechanical vibrator 28, or the heating elements 128A/128B, along with the power supply 22, function generator 24 and amplifier 26 (e.g., low noise precision instrumentation amplifier by Analog Devices; AMP01), can be contained within a single housing that is portable, thereby allowing the apparatus 20 or 120 to be used outside of the catheter laboratory.

Without further elaboration, the foregoing will so fully illustrate my invention that others may, by applying current or future knowledge, readily adopt the same for use under various conditions of service.

I claim:

1. A method for preventing, or reducing the likelihood of, renal failure in a living being due to the presence of a contrast media agent in the bloodstream of the being, said method comprising the step of reducing the viscosity of the blood in the kidney.

2. The method of claim 1 wherein said step of reducing the viscosity of the blood in the kidney comprises applying vibratory energy to the kidney.

3. The method of claim 2 wherein said vibratory energy comprises ultrasonic energy.

4. The method of claim 2 wherein said step of applying vibratory energy comprises shaking thromboses formed in the microvessels of the kidney.

5. The method of claim 2 wherein said step of applying vibratory energy comprises breaking blood clots formed in the microvessels of the kidney.

6. The method of claim 1 wherein said step of reducing the viscosity of the blood in the kidney comprises applying heat to the kidney.

7. An apparatus for preventing, or reducing the likelihood of, renal failure in a living being due to the presence of a contrast media agent in the bloodstream of the being, said apparatus comprising a vibrator for producing vibratory energy when energized, said apparatus being locatable with respect to the body of the being to provide the vibratory energy to the being to reduce the viscosity of the blood in the being's kidney.

8. The apparatus of claim 7 wherein said vibrator is a mechanical vibrator that is disposed on the back of the being in the vicinity of the kidney to reduce the viscosity of the blood in the kidney.

9. The apparatus of claim 8 further comprising a function generator coupled to said mechanical vibrator for generating the frequency of the vibratory energy.

10. The apparatus of claim 9 further comprising a power source coupled to said function generator for energizing the function generator.

11. The apparatus of claim 10 further comprising an amplifier coupled between said function generator and said mechanical vibrator for amplifying the frequency of the vibratory energy that is supplied to said mechanical vibrator.

12. The apparatus of claim 7 wherein said vibrator is an ultrasonic device.

13. The apparatus of claim 11 wherein said mechanical vibrator is an ultrasonic device.

14. An apparatus for preventing, or reducing the likelihood of, renal failure in a living being due to the presence of a contrast media agent in the bloodstream of the being, said apparatus comprising a heater for producing heat energy when energized, said apparatus being locatable with respect to the body of the being to provide the heat energy to the being to reduce the viscosity of the blood in the being's kidney.

15. The apparatus of claim 14 wherein said heater is disposed on the back of the being in the vicinity of the kidney to reduce the viscosity of the blood in the kidney.

16. The apparatus of claim 15 further comprising a power supply coupled to said heater for energizing said heater.
